(19) United States
(12) Patent Application Publication
(10) Pub. No.: US 2014/0004010 A1
(43) Pub. Date: Jan. 2, 2014

(54) AIR-PURIFYING DEVICE FOR VEHICLE

(71) Applicants: Kazuhiro Sugimoto, Susono-shi (JP); Koichi Hoshi, Susono-shi (JP); Takashi Watanabe, Gotenba-shi (JP); Yoshihisa Shinoda, Susono-shi (JP); Ippei Fukutomi, Machida-shi (JP)

(72) Inventors: Kazuhiro Sugimoto, Susono-shi (JP); Koichi Hoshi, Susono-shi (JP); Takashi Watanabe, Gotenba-shi (JP); Yoshihisa Shinoda, Susono-shi (JP); Ippei Fukutomi, Machida-shi (JP)

(21) Appl. No.: 14/001,593

(22) PCT Filed: Mar. 23, 2011
(86) PCT No.: PCT/JP2011/056911

§ 371 Date: Aug. 26, 2013

(51) Int. Cl. A61L 9/015

(52) U.S. Cl. CPC A61L 9/015
USPC 422/123

(57) ABSTRACT

The present invention relates to an air-purifying device for a vehicle and an object thereof is to provide a DOR (Direct Ozone Reduction) system which can favorably purify ozone even in a normal temperature range and can suppress decrease in the cooling performance of a radiator caused by coating.

The device comprises an on-vehicle component arranged at a spot in contact with the air during travel of the vehicle, and an ozone purifier provided on the on-vehicle component and capable of purifying ozone, wherein the ozone purifier includes at least one of organic metal complexes having manganese, iron, cobalt, nickel, copper, ruthenium, rhodium or palladium as a central metal. The ozone purifier preferably includes activated carbon.

### TECHNICAL FIELD

The present invention relates to an air-purifying device for a vehicle and particularly to an air-purifying device for a vehicle capable of purifying ozone in atmospheric air.

### BACKGROUND ART

Ozone, which causes photochemical smog, is generated by a photochemical reaction of HC and NOx contained in exhaust gases from automobiles and factories. Therefore, reducing the amount of HC and NOx emissions from automobiles is an efficient way to suppress the production of ozone and the occurrence of photochemical smog. On the other hand, purifying ozone in the air directly can be one of the ways to prevent the occurrence of photochemical smog. By purifying ozone as a product as well as reducing the amount of emissions of HC and NOx as reactants, the occurrence of photochemical smog can be prevented more effectively. From such a point of view, an automobile comprising an air-purifying device for a vehicle capable of directly purifying ozone in atmospheric air has been put into practical use in some places including California in the United States of America. This air-purifying device for a vehicle, particularly, is called a DOR (Direct Ozone Reduction) system.

For example, Patent Literature 1 discloses a DOR system in which a metal oxide such as manganese dioxide is supported by an on-vehicle component. An on-vehicle component such as a radiator is disposed at a spot in contact with atmospheric air during travel of the vehicle, and manganese dioxide has a function of converting ozone contained in the air into other substances such as oxygen, and purifying ozone. Therefore, according to the DOR system disclosed in Patent Literature 1, ozone in the atmospheric air can be directly purified during travel of the vehicle.

### CITATION LIST

### Patent Literature

Patent Literature 1: National Publication of International Patent Application No. 2002-514966

### SUMMARY OF INVENTION

However, a problem of manganese dioxide is that its function for purifying ozone decreases in a normal temperature range. This problem will be described by referring to FIG. 7. FIG. 7 is a graph illustrating a relationship between an ozone purification rate (%) of manganese dioxide and a wind velocity (m/s). This graph was created by preparing a manganese dioxide test piece and then measuring an ozone concentration in the rear of the test piece when air having an ozone concentration of 0.2 ppm is made to pass from the front toward the rear of the test piece at different speeds. As illustrated in FIG. 7, in a case of the test piece temperature of 25° C. (degrees Celsius), the ozone purification rate decreases more than in a case of that of 75° C. Therefore, according to FIG. 7, it can be understood that the ozone purification function of manganese dioxide is not fully exerted in a normal temperature range where an engine is in warming-up operation.

Another problem is that if manganese dioxide is supported by the on-vehicle component, the temperature of the on-vehicle component rises. For example, if a cooling fin of a radiator is coated with manganese dioxide, the cooling fin should be covered by a manganese dioxide layer with lower heat conductivity than that of the cooling fin. Thus, high heat conductivity specific to the cooling fin decreases and heat conductivity of the entire radiator decreases. Therefore, the cooling performance of the radiator decreases.

The present invention has been made in view of the above-described problems. It is an object to provide a DOR system which can favorably purify ozone even in a normal temperature range and can suppress decrease in the cooling performance of the radiator caused by coating.

### Means for Solving the Problem

To achieve the above-mentioned purpose, a first aspect of the present invention is an air-purifying device for a vehicle, comprising:

an on-vehicle component arranged at a spot in contact with the air during travel of a vehicle; and

an ozone purifier provided on the on-vehicle component and capable of purifying ozone, wherein

the ozone purifier includes at least one of organic metal complexes having manganese, iron, cobalt, nickel, copper, ruthenium, rhodium or palladium as a central metal.

A second aspect of the present invention is the air-purifying device for a vehicle according to the first aspect, wherein

the organic metal complex is a salen complex represented by the following formula (I), a porphyrin complex represented by the following formula (II), a phthalocyanine complex represented by the following formula (ID) or a phenanthroline complex represented by the following formula (IV):

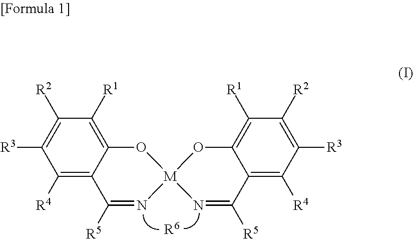

wherein M represents manganese, iron, cobalt, nickel, copper, ruthenium, rhodium or palladium, R¹ to R⁵ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, and R⁶ represents a linear or branched alkylene group having 2 to 8 carbon atoms or a cycloalkylene group having 3 to 8 carbon atoms or represents a general formula —(CH₂)ₚ—NR⁷—(CH₂)_{q}— (wherein R⁷ is a hydrogen atom or a methyl group, and p and q are each an integer of 1 to 4);

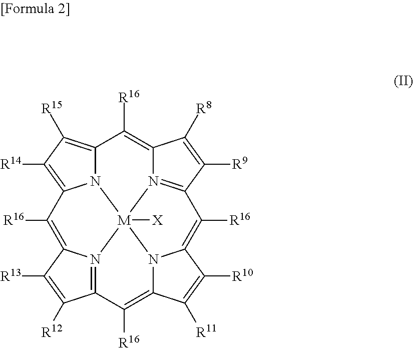

wherein M represents manganese, iron, cobalt, nickel, copper, ruthenium, rhodium or palladium, R⁸ to R¹⁵ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, R¹⁶ represents a hydrogen atom or a phenyl group which may be substituted, and X represents a halogen atom, an isothiocyanato group, imidazole and a derivative thereof, pyridine and a derivative thereof, aniline and a derivative thereof or histidine and a derivative thereof;

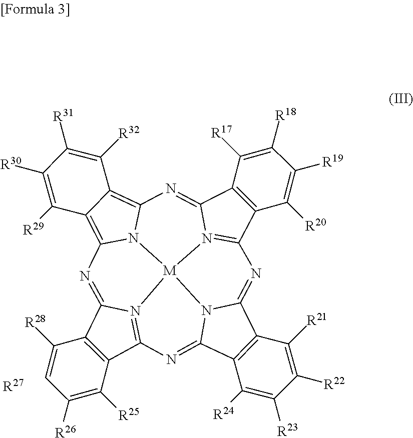

wherein M represents manganese, iron, cobalt, nickel, copper, ruthenium, rhodium or palladium, R¹⁷ to R³² each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group; and

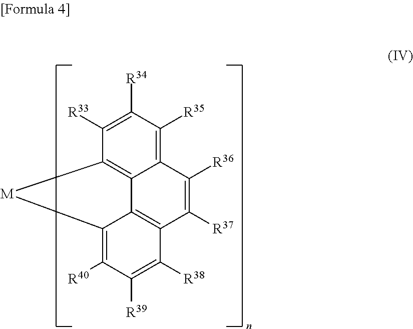

wherein M represents manganese, iron, cobalt, nickel, copper, ruthenium, rhodium or palladium, R³³ to R⁴⁰ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, and n represents a natural number.

A third aspect of the present invention is the air-purifying device for a vehicle according to the first or second aspect of the present invention, wherein the organic metal complex is a picket fence porphyrin complex represented by the following formula (II-a) or (II-b):

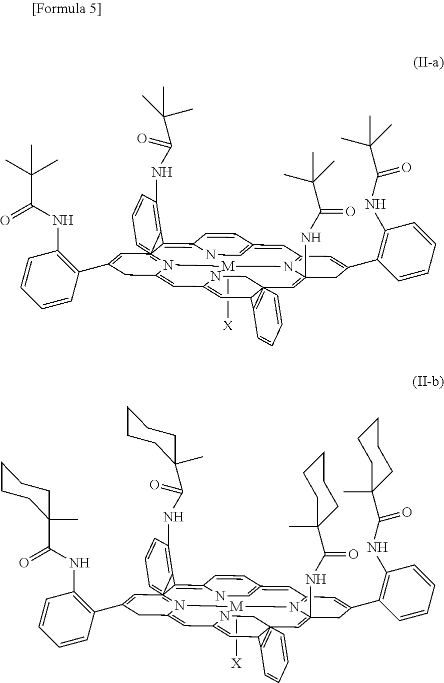

wherein M represents manganese, iron, cobalt, nickel, copper, ruthenium, rhodium or palladium, and X represents a halogen atom, an isothiocyanato group, imidazole and a derivative thereof, pyridine and a derivative thereof, aniline and a derivative thereof or histidine and a derivative thereof.

A forth aspect of the present invention is the air-purifying device for a vehicle according to any one of the first to the third aspect of the present invention, wherein the ozone purifier further includes activated carbon.

### Advantageous Effects of Invention

According to the first to fourth aspects of the inventions, a DOR system in which ozone purifying can be favorably performed even in the normal temperature range and moreover, decrease in the cooling performance of the radiator caused by coating can be suppressed can be provided.

### BRIEF DESCRIPTION OF DRAWINGS

FIG. 1 is a schematic diagram illustrating a configuration of a vehicle on which an air-purifying device according to the embodiment of the present invention is applied.

FIG. 2 is a graph illustrating an ozone purification rate of the salen complex.

FIG. 3 is a graph illustrating a relationship between an ozone exposure time (hr) and the ozone purification rate (%).

FIG. 4 are diagrams for describing the ozone purifying mechanism.

FIG. 5 is a graph illustrating a relationship between the ozone exposure time(s) and the ozone purification rate (%).

FIG. 6 is a diagram for describing a structure of the picket fence porphyrin complex.

FIG. 7 is a graph illustrating a relationship between an ozone purification rate (%) of manganese dioxide and a wind velocity (m/s).

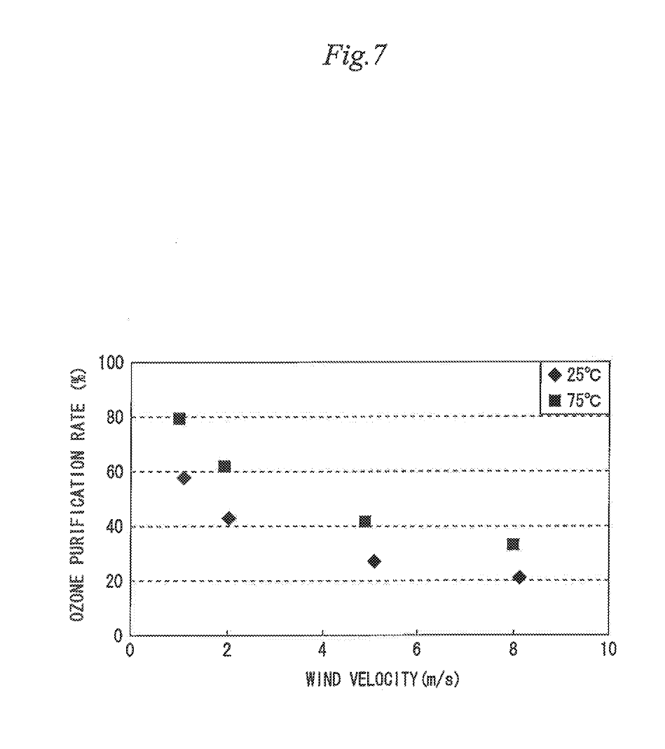

### DESCRIPTION OF EMBODIMENTS

### Configuration of Air-Purifying Device for a Vehicle

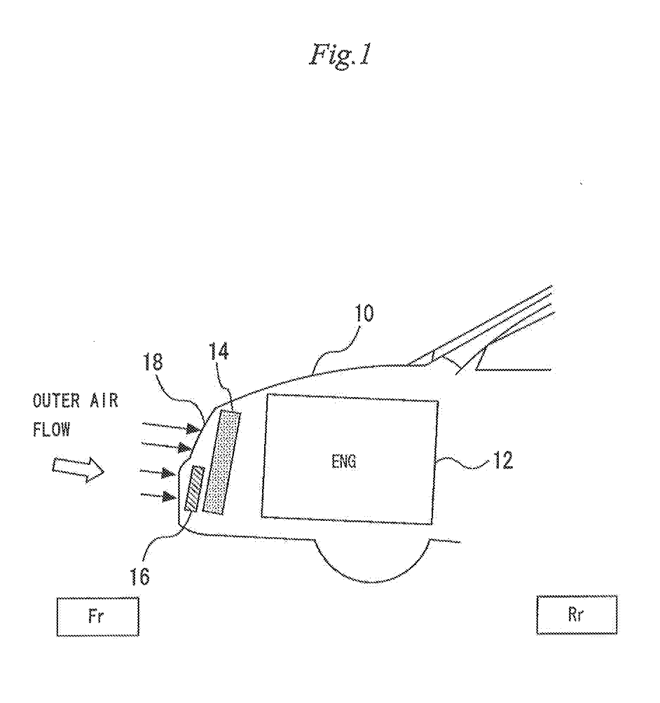

An embodiment of the present invention will be described below by referring to FIGS. 1 to 6. FIG. 1 is a schematic diagram illustrating a configuration of a vehicle on which an air-purifying device according to this embodiment is applied. A vehicle 10 comprises an internal combustion engine 12 as a power device. An exhaust gas discharged from the internal combustion engine 12 includes HC and NOx. Ozone is generated by photochemical reactions using HC and NOx as reactants. Thus, by mounting an air-purifying device on the vehicle 10 comprising the internal combustion engine 12 and by purifying ozone in the air during travel of the vehicle 10, an influence of the vehicle 10 on the environment can be reduced.

In the vehicle 10, a radiator 14 for cooling coolant water to be circulated in the internal combustion engine 12 is arranged on the front of the internal combustion engine 12. On the front of the radiator 14, a capacitor 16 for an air conditioner is mounted. As illustrated by an arrow in FIG. 1, during travel of the vehicle 10, atmospheric air is taken in through a bumper grill 18 on a front surface of the vehicle 10, and the taken-in air passes through the capacitor 16 and the radiator 14 in this order and is discharged to the rear.

A fin (not shown) is provided on a core of the radiator 14. In the air-purifying device of this embodiment, the surface of this fin is coated with an ozone purifier containing at least one of organic metal complexes having manganese, iron, cobalt, nickel, copper, ruthenium, rhodium or palladium as a central metal. Thus, first, the organic metal complex that can be preferably used for this ozone purifier will be described.

### [Organic Metal Complex]

Examples of an organic metal complex that can be preferably used for the ozone purifier include, first, a salon complex represented by the following formula (I):

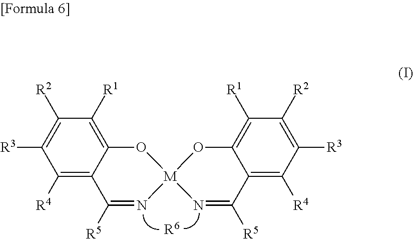

wherein M represents manganese, iron, cobalt, nickel, copper, ruthenium, rhodium or palladium, R¹ to R⁵ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, and R⁶ represents a linear or branched alkylene group having 2 to 8 carbon atoms or a cycloalkylene group having 3 to 8 carbon atoms or represents a general formula —(CH₂)ₚ—NR⁷—(CH₂)_{q}— (wherein R⁷ is a hydrogen atom or a methyl group, and p and q are each an integer of 1 to 4).

Here, examples of the alkyl group having 1 to 8 carbon atoms include a methyl group, an ethyl group, a n-propyl group, an i-propyl group, a n-butyl group, an i-butyl group, a sec-butyl group a t-butyl group, a t-pentyl group, an i-octyl group, a t-octyl group, and a 2-ethylhexyl group. Examples of the alkenyl group having 2 to 8 carbon atoms include a 1-propenyl group, a 2-propenyl group, a 2-methyl-1-propenyl group, a 2-methyl-2-propenyl group, a 1-butenyl group, a 2-butenyl group, and a 3-butenyl group. Examples of the acyl group having 2 to 8 carbon atoms include an acetyl group, a propanoyl group, a butanoyl group, a pentanoyl group, and a benzoyl group.

Moreover, examples of the linear or branched alkylene group having 2 to 8 carbon atoms include an ethylene group, a propylene group, a butylene group, a pentamethylene group, a hexamethylene group, an octamethylene group, and a 2,2-dimethyl-1,3-propylene group. Moreover, examples of the cycloalkylene group having 3 to 8 carbon atoms include a cycloheptyl group, a cyclohexyl group, and a cyclopentyl group.

Moreover, examples of the organic metal complex that can be preferably used for the ozone purifier also include a porphyrin complex represented by the following formula (II):

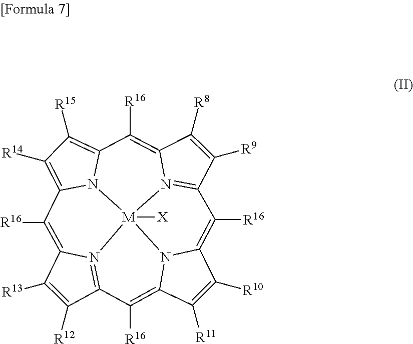

wherein M represents manganese, iron, cobalt, nickel, copper, ruthenium, rhodium or palladium, R⁸ to R¹⁵ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, R¹⁶ represents a hydrogen atom or a phenyl group which may be substituted, and X represents a halogen atom, an isothiocyanato group, imidazole and a derivative thereof, pyridine and a derivative thereof aniline and a derivative thereof or histidine and a derivative thereof.

Here, as the alkyl group having 1 to 8 carbon atoms, the alkenyl group having 2 to 8 carbon atoms, and the acyl group having 2 to 8 carbon atoms, those listed in the description of R¹ to R⁵ are applicable.

Examples of the derivative of imidazole include methyl imidazole, ethyl imidazole, propyl imidazole, dimethyl imidazole, and benzimidazole. Examples of the derivative of pyridine include methyl pyridine, methyl pyridylacetate, nicotinamide, pyridazine, pyrimidine, pyrazine, and triazine. Examples of the derivative of aniline include aminophenol and diaminobenzene, Examples of the derivative of histidine include histidine methyl ester, and histamine.

Among the porphyrin complexes represented by the above-described formula (II), picket fence porphyrin complexes represented by the following formula (II-a) or a formula (II-b) can be used particularly preferably. The detailed reason for that will be described later, but by having a picket fence structure, the porphyrin complexes can be used particularly preferably, since coordination of substances other than ozone to the central metal of the porphyrin complex can be favorably suppressed.

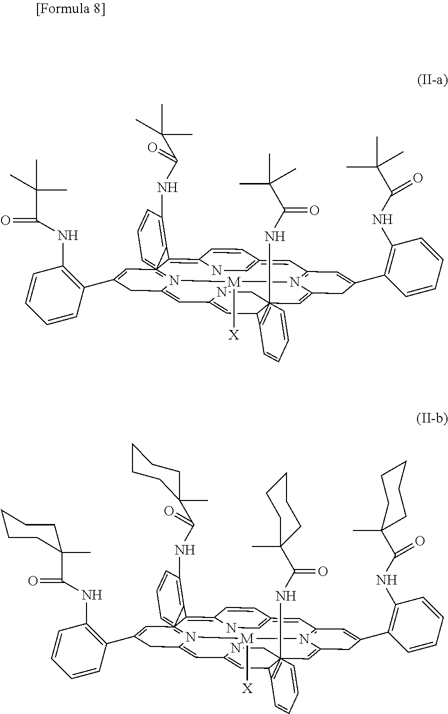

wherein M represents manganese, iron, cobalt, nickel, copper, ruthenium, rhodium or palladium, and X represents a halogen atom, isothiocyanato group, imidazole and a derivative thereof, pyridine and a derivative thereof, aniline and a derivative thereof or histidine and a derivative thereof.

Here, as the derivative of imidazole, the derivative of pyridine, the derivative of aniline, and the derivative of histidine, those listed in the description of the above-described formula (II) are applicable.

Moreover, examples of the organic metal complex that can be preferably used for the ozone purifier also include a phthalocyanine complex represented by the following formula (III):

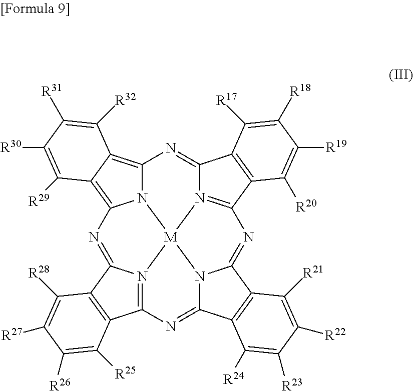

wherein M represents manganese, iron, cobalt, nickel, copper, ruthenium, rhodium or Palladium, R¹⁷ to R³² each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group.

Here, as the alkyl group having 1 to 8 carbon atoms, the alkenyl group having 2 to 8 carbon atoms, and the acyl group having 2 to 8 carbon atoms, those listed in the description of R¹ to R⁵ are applicable.

Moreover, examples of the organic metal complex that can be preferably used for the ozone purifier also include a phenanthroline complex represented by the following formula (IV):

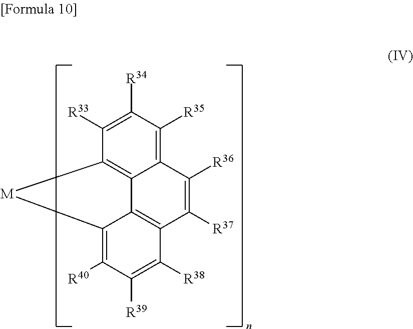

wherein M represents manganese, iron, cobalt, nickel, copper, ruthenium, rhodium or palladium, R³³ to R⁴⁰ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, and n represents a natural number.

Here, as the alkyl group having 1 to 8 carbon atoms, the alkenyl group having 2 to 8 carbon atoms, and the acyl group having 2 to 8 carbon atoms, those listed in the description of R¹ to R⁵ are applicable.

### [Ozone Purifying Ability of Organic Metal Complex]

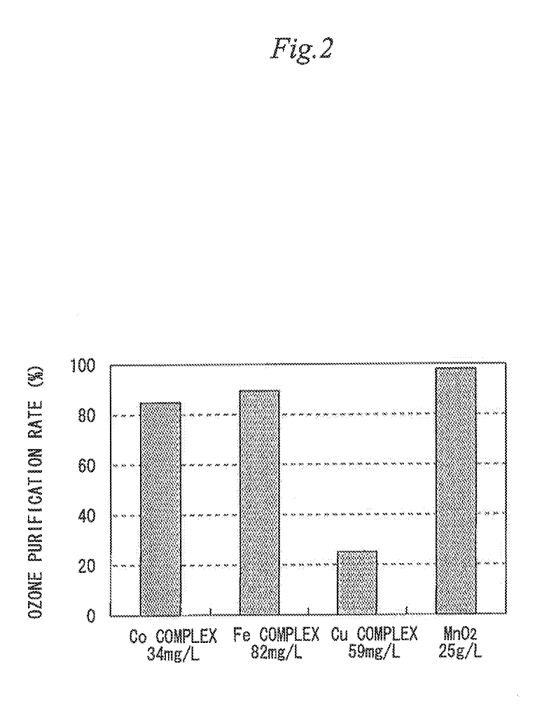

FIG. 2 is a graph illustrating an ozone purification rate of the salen complex in the above-described formula (I). This graph was created by preparing the respective salen complexes (central metal is cobalt, iron and copper) on the surface of radiators, and by measuring an ozone concentration in the rear of these radiators, under the conditions of a radiator bed temperature of 80° C. and a relative humidity of 60% (25° C.), while letting air having an ozone concentration of 0.5 ppm to pass through from the front to the rear of them. In each radiator, a support amount per unit volume of the salen complex was set to Co complex: 34 mg/L, Fe complex: 82 mg/L, Cu complex: 59 mg/L.

As illustrated in FIG. 2, each salen complex has an ability for purifying ozone, and particularly the Co complex and the Fe complex have the abilities for purifying ozone equal to manganese dioxide (support amount per unit volume: 25 g/L) for comparison. From this result, it can be understood that though there is a difference in ozone purifying property depending on the type of the central metal, the organic metal complex shows the ability for purifying ozone equal to that of manganese dioxide with a smaller support amount than that of manganese dioxide. When the bed temperature among the above-described conditions was changed to 25 to 120° C. and the ozone concentration was measured, the same result as this graph was obtained.

From this result, it can be understood that the organic metal complex shows a favorable ability for purifying ozone with a small support amount, Moreover, it can be understood that the organic metal complex shows a favorable ability for purifying ozone in a wide temperature range including the normal temperature range. Therefore, the use of the organic metal complex as the ozone purifier makes it possible to suppress the temperature rise of the radiator caused by support of the ozone purifier thereby suppress the decreases in the cooling performance of the radiator, and also possible to purify ozone favorably even in the normal temperature range. The present inventors infer that the above-described result was obtained because ligands of the organic metal complex act as electron donor group thereby causes an activation of the ozone purifying ability of the central metal.

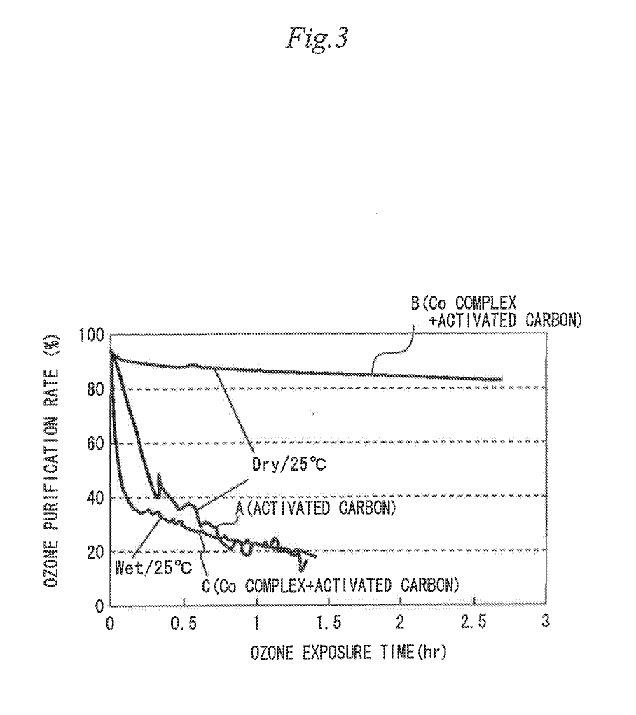

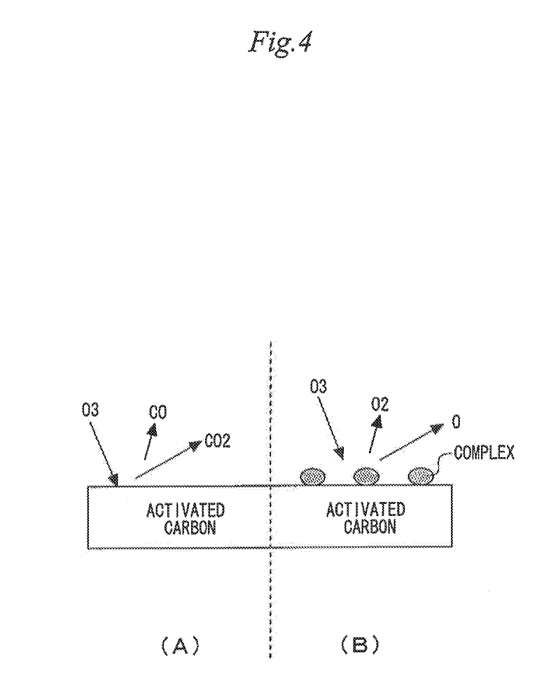

Subsequently, an activated carbon that can be preferably used, along with the above-described organic metal complex, for the ozone purifier will be described by referring to FIGS. 3 and 4. The activated carbon is available at moderate. price and known to show a high ability for ozone purifying in the normal temperature zone. On the other hand, the activated carbon has a characteristic that its function for purifying ozone can easily decrease over time.

However, according to the findings of the present inventors, the time degradation of the activated carbon has revealed to be suppressible by combining the above-described organic metal complex with the activated carbon. FIG. 3 is a graph illustrating a relationship between an ozone exposure time (hr) and the ozone purification rate (%). This graph was created by preparing a radiator coated only with an activated carbon (radiator A) and radiators (radiators B and C) coated with the activated carbon and the salen complex of the above-described formula (I), separately, and by measuring the ozone concentration in the rear of each radiator at each exposure time, while letting air having an ozone concentration of 110 ppm to pass through the front to the rear of the radiator under a Wet condition (a moisture concentration of 2%, a humidity of worth 60%) or a Dry condition)) in a state where the radiator bed temperature is kept at 25° C. In each radiator, a support amount per unit volume of the activated carbon was set to 25 g/L. Moreover, in the radiators B and C, the support amount of the salen complex was prepared so as to become 0.4 wt % with respect to the activated carbon.

As illustrated in FIG. 3, the ozone purification rate of the radiator A decreased as the exposure time elapsed. On the other hand, the ozone purification rate of the radiator B was kept at a high level over a long period of time. Here, the difference between the radiator A and the radiator B is presence of coating of the organic metal complex. Therefore, from this result, it can be understood that an ozone purifier showing a favorable ability for ozone purifying over a long period of time can be obtained by combining the organic metal complex with the activated carbon. The present inventors infer that such a result is due to a purifying mechanism illustrated in FIG. 4.

FIG. 4 are diagrams for describing the ozone purifying mechanism. FIG. 4(A) corresponds to the radiator A in FIG. 3 and FIG. 4(B) to the radiator B in FIG. 3. In the activated carbon, reaction in the following formulas (1) to (4) progress:

O₃→O₃⁻ (1)

O₃⁻→O₂+O⁻ (2)

C+O→CO (3)

C+2O→CO₂ (4)

The reaction in the above-described formula (1) or (2) is a reaction where ozone is decomposed in the pores of the activated carbon (ozone decomposition reaction). This ozone decomposition reaction progresses specifically such that an ozone molecule enters into the pore of the activated carbon, and electrons are donated from the activated carbon in this pore. The reaction in the above-described formula (3) or (4) is a reaction in which a carbon atom constituting the activated carbon is consumed (carbon consumption reaction). This carbon consumption reaction progresses in the activated carbon on the radiator A. Therefore, in the radiator A, as illustrated in FIG. 4(A), the carbon atom in the activated carbon becomes CO and CO₂, and its pore structure changes over time, whereby function for purifying decreases.

On the other hand, in the organic metal complex, the reaction in the following formulas (5) to (7) progress:

O₃→O₃⁻ (5)

O₃⁻→O₂+O⁻ (6)

O⁻+O₃⁻→2CO₂ (7)

The reaction in the above-described formula (5) or (6) is a reaction progressing on the central metal of the organic metal complex and expressed as the same reaction formula in the above-described formula (1) or (2). The reaction in the above-described formula (7) is a reaction (complex reaction) progressing on the central metal of the organic metal complex similarly to the above-described formula (5) or (6). This complex reaction can use not only the reaction of the above-described formula (5) or (6) but also O₃⁻ and O⁻ generated by the reactions in the above-described formula (1) or (2). Therefore, in the radiator B, as illustrated in FIG. 4(B), the above-described carbon consumption reaction is suppressed.

Returning to FIG. 3, again, the description will be continued. As illustrated in FIG. 3, the ozone purification rate of the radiator C decreased with the elapse of the exposure time. On the other hand, the ozone purification rate of the radiator B was kept at a high level over a long period of time. Here, the difference between the radiator B and the radiator C is a difference in the moisture amount condition (Wet condition or Dry condition) in the air made to pass through the radiator. Therefore, a possibility is shown from this result that the ozone purifying function of the organic metal complex is obstructed in the Wet condition.

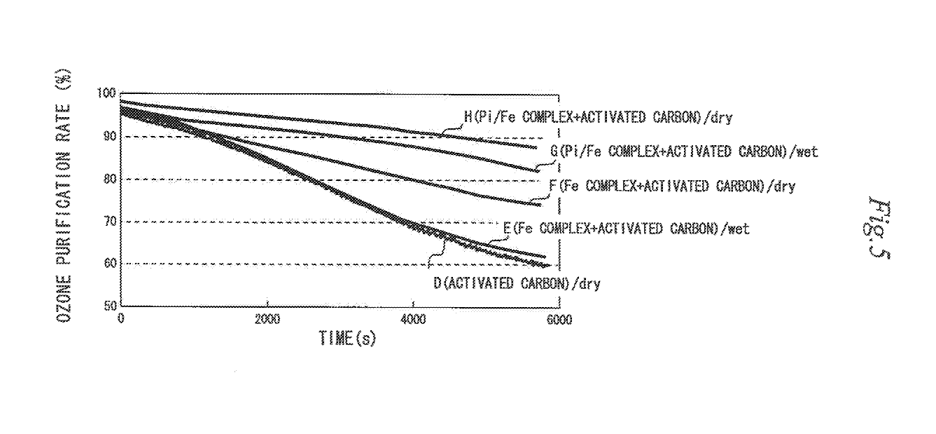

However, according to the findings of the present inventors, the decrease in the ozone purifying function of the organic metal complex has revealed to be suppressible, even under the Wet condition, by using the picket fence porphyrin complexes represented by the above-described formula (II-a) or (II-b) among the above-described organic metal complexes. FIG. 5 is a graph illustrating a relationship between the ozone exposure time (s) and the ozone purification rate (%). This graph was created by preparing a radiator (the radiator D) coated only with the activated carbon, radiators (the radiators E and F) coated with the activated carbon and the porphyrin complex (the central metal is iron) of the above-described formula (II), and radiators (the radiators G and H) coated with the activated carbon and the picket fence porphyrin complex (the central metal is iron) of the above-described formula (II-a), separately, and by measuring the ozone concentration in the rear of each radiator at each exposure time, while letting air having an ozone concentration of 130 ppm to pass through the front to the rear of the radiator (under a Wet condition (moisture concentration of 2%, a humidity of worth 60%) or a Dry condition)) in a state where the radiator bed temperature is kept at 80° C.

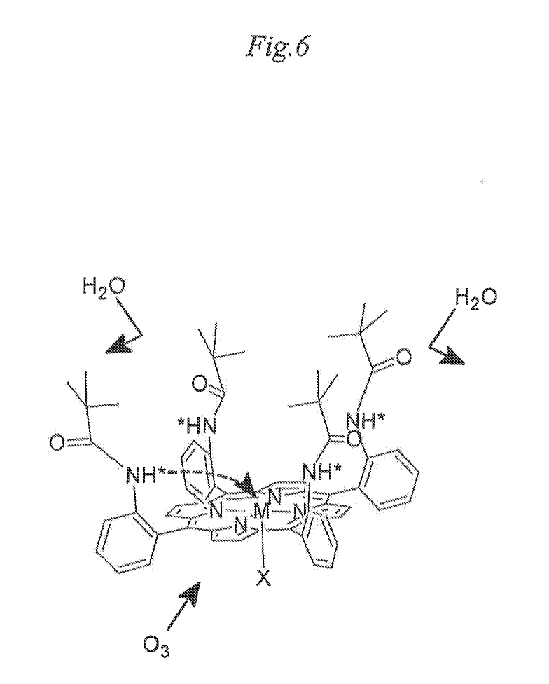

As illustrated in FIG. 5, the ozone purification rate of the radiators G and H were kept at a high level over a long period of time. Here, the difference between the radiator G and the radiator H is a difference in the moisture amount condition (Wet condition or Dry condition) in the air made to pass through the radiator. Therefore, it can be understood from this result that the ozone purifier showing a favorable ability for ozone purifying over a long period of time can be obtained by using the picket fence porphyrin complex. The present inventors infer that such a result is due to the characteristic of the structure of the picket fence type. This inference will be described by referring to FIG. 6.

FIG. 6 is a diagram for describing a structure of the picket fence porphyrin complex. If the organic metal complex is used, a substance other than ozone such as a water molecule, a hydrogen peroxide molecule generated from the reaction between water and ozone, a proton generated from hydrogen peroxide, or a superoxide can coordinate onto the central metal. If a substance other than ozone coordinates onto the central metal, it is likely that the coordination probability of ozone decreases by that portion. Thus, it is likely that the ozone purifying function of the organic metal complex is obstructed. In this regard, the picket fence complex can take a structure such that, as illustrated in FIG. 6, a hydrogen atom (H*) on an amide residue coordinates onto the central metal and thus, the picket fence covers the central metal. Therefore, decrease in the coordination probability of ozone caused by the substance other than ozone can be favorably suppressed.

In FIG. 5, the reason why the ozone purification rate of the radiator F was kept at a high level over a long period of time, though the level is lower than those of the radiators G and H, is similar to the reason for the result of the radiator B in FIG. 3 Moreover, the reason why the ozone purification rate of the radiators D and E lowered with elapse of the exposure time is similar to the reason for the results of the radiators A and C in FIG. 3.

### DESCRIPTION OF REFERENCE NUMERALS

10 vehicle

12 internal combustion

14 radiator

16 capacitor

18 bumper grill

1. An air-purifying device for a vehicle comprising:
an on-vehicle component arranged at a spot in contact with the air during travel of a vehicle; and
an ozone purifier provided on the on-vehicle component and capable of purifying ozone, wherein
the ozone purifier includes at least one of organic metal complexes having manganese, iron, cobalt, nickel, copper, ruthenium, rhodium or palladium as a central metal, and wherein
the organic metal complex is a salen complex represented by the following formula (I), a porphyrin complex represented by the following formula (II), a phthalocyanine complex represented by the following formula (III) or a phenanthroline complex represented by the following formula (IV): 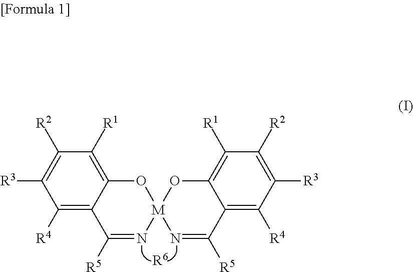
wherein M represents manganese, iron, cobalt nickel, copper, ruthenium, rhodium or palladium, R¹ to R⁵ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon, atoms or a nitro group, and R⁶ represents a linear or branched alkylene group having 2 to 8 carbon atoms or a cycloalkylene group having 3 to 8 carbon atoms or represents a general formula —(CH₂)ₚ—NR⁷—(CH₂)_{q}— (wherein R⁷ is a hydrogen atom or a methyl group, and p and q are each an integer of 1 to 4): 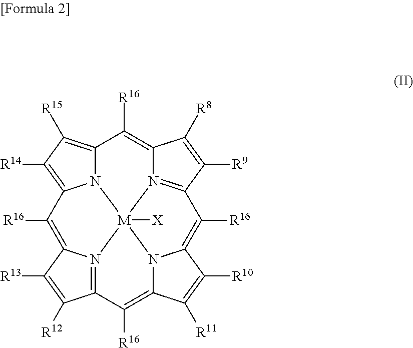
wherein M represents manganese, iron, cobalt, nickel, copper, ruthenium, rhodium or palladium, R⁸ to R¹⁵ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, R¹⁶ represents a hydrogen atom or a phenyl group which may be substituted, and X represents a halogen atom, an isothiocyanato group, imidazole and a derivative thereof, pyridine and a derivative thereof, aniline and a derivative thereof or histidine and a derivative thereof; 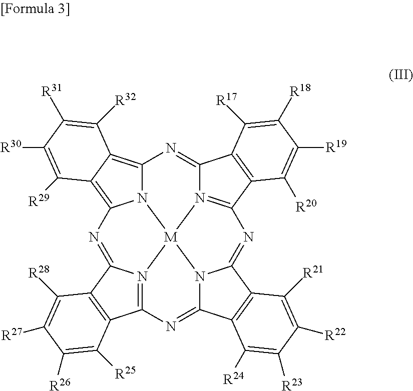
Wherein M represents manganese, iron, cobalt, nickel, copper, ruthenium, rhodium or palladium, R¹⁷ to R³² each independently represent a hydrogen atom, a halogen atom, an alkyl group having 1 to 8 carbon atoms, an alkenyl group haying 2 to 8 carbon atoms, a formyl group, a carboxyl group, an acyl group having 2to 8 carbon atoms or a nitro group; and 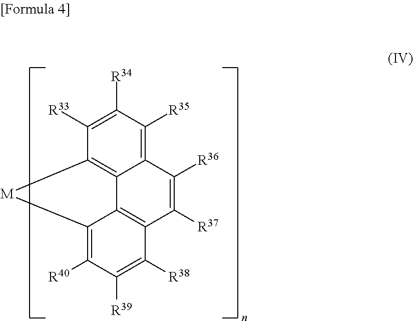
wherein M represents manganese, iron, cobalt, nickel, copper, ruthenium, rhodium or palladium, R³³ to R⁴⁰ each independently represent a hydrogen atom, a halogen atom, an alkyl group having 1 to 8 carbon atoms, an alkenyl group having 2 to 8 carbon atoms, a formyl group, a carboxyl group, an acyl group having 2 to 8 carbon atoms or a nitro group, and n represents, a natural number.

2. (canceled)

3. The air-purifying device for a vehicle according to claim 1, wherein the organic metal complex is a picket fence porphyrin complex represented by the following formula (II-a) or (II-b): 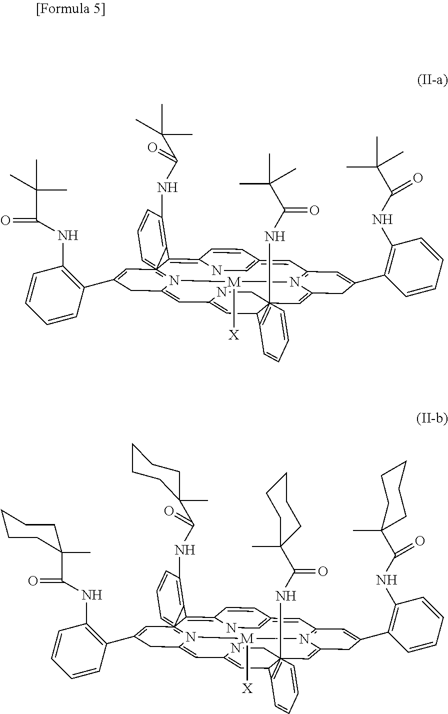
wherein M represents manganese, iron, cobalt, nickel, copper, ruthenium, rhodium or palladium, and X represents a halogen atom, an isothiocyanato group, imidazole and a derivative thereof, pyridine and a derivative thereof, aniline and a derivative thereof or histidine and a derivative thereof.

4. The air-purifying device for a vehicle according to claim 1, wherein the ozone purifier further includes activated carbon.

5. The air-purifying device for a vehicle according claim 3, wherein the ozone purifier further includes activated carbon.
